(19) United States
(12) Plant Patent Application Publication
(10) Pub. No.: US 2015/0156939 P1
(43) Pub. Date: Jun. 4, 2015

(54) Hop plant named 'ADHA 871'

(50) ;

(71) Applicant: Jason Perrault, Toppenish, WA (US)

(72) Inventor: Jason Perrault, Toppenish, WA (US)

(21) Appl. No.: 14/121,499

(22) Filed: Sep. 12, 2014

Related U.S. Application Data
(60) Provisional application No. 61/960,260, filed on Sep. 13, 2013.

(51) Int. Cl. A01H 5/00

(52) U.S. Cl. PLT/236

(57) ABSTRACT

A new hop plant named ADHA 871 is disclosed. The cones of ADHA 871 mature in mid to late September in Moxee, Wash., and yield a crop of 2200 to 2300 pounds per acre. ADHA 871 is used for its aromatic quality.

### CROSS-REFERENCE TO RELATED APPLICATIONS

This application claims priority of co-pending provisional application serial number 61/960,260.

### STATEMENT REGARDING FEDERALLY SPONSORED RESEARCH OR DEVELOPMENT

None

### GENUS AND SPECIES

Humulus lupulus

### VARIETY DENOMINATION

ADHA 871

### BACKGROUND OF THE INVENTION

‘ADHA 871’ is a product of a controlled breeding program carried out by the inventor in the Yakima Valley of Washington State. ADHA 871 was one of several seedlings resulting from cross number 08-071 made in 2008 with female parent ADHA 538 (not patented) which was open pollinated with male hops plants. A single plant of ADHA 871 was selected in 2009. In 2011 that single plant was expanded by asexually propagation into a large scale test plot in Moxee, Wash. for further observation and evaluation. An evaluation of that test plot shows that ADHA 871 has been observed to retain its distinctive characteristics after asexual propagation, and has remained true to type.

### BRIEF DESCRIPTION OF THE PHOTOGRAPHS

FIG. 1 illustrates a mature ADHA 871 hop plant grown on a trellis;

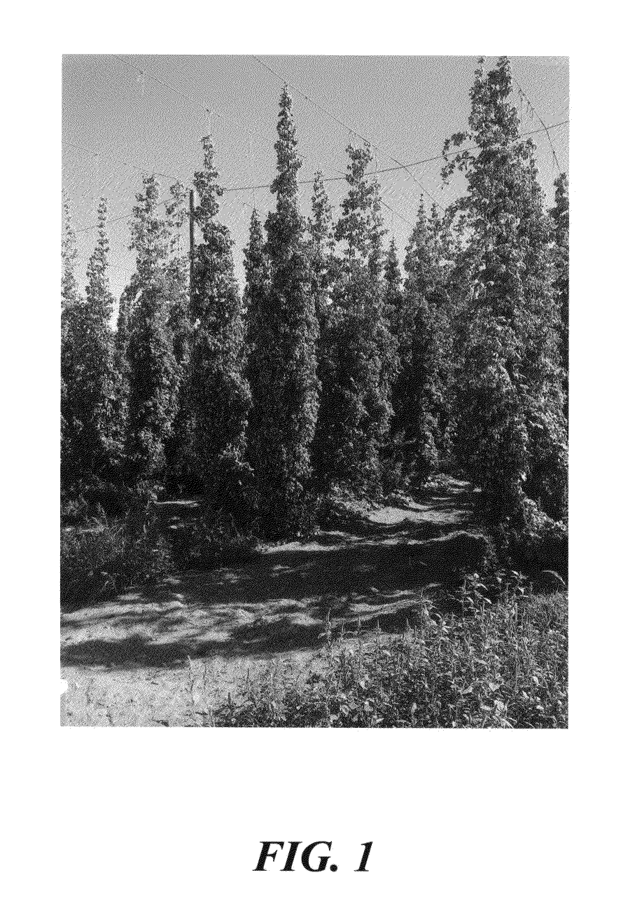

FIG. 2 illustrates whole cones and cross sections of cones of the ADHA 871 hop plant;

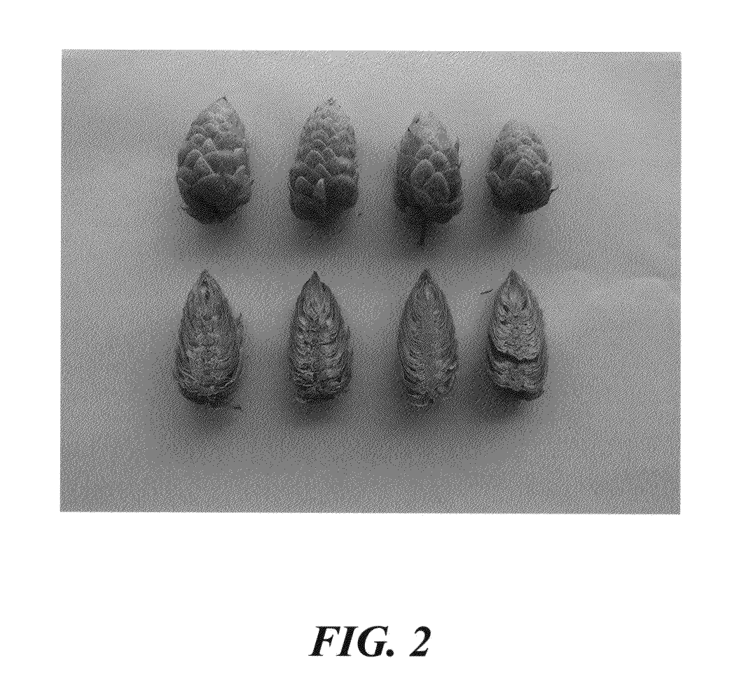

FIG. 3 illustrates the bine of a mature ADHA 871 hop plant; and.

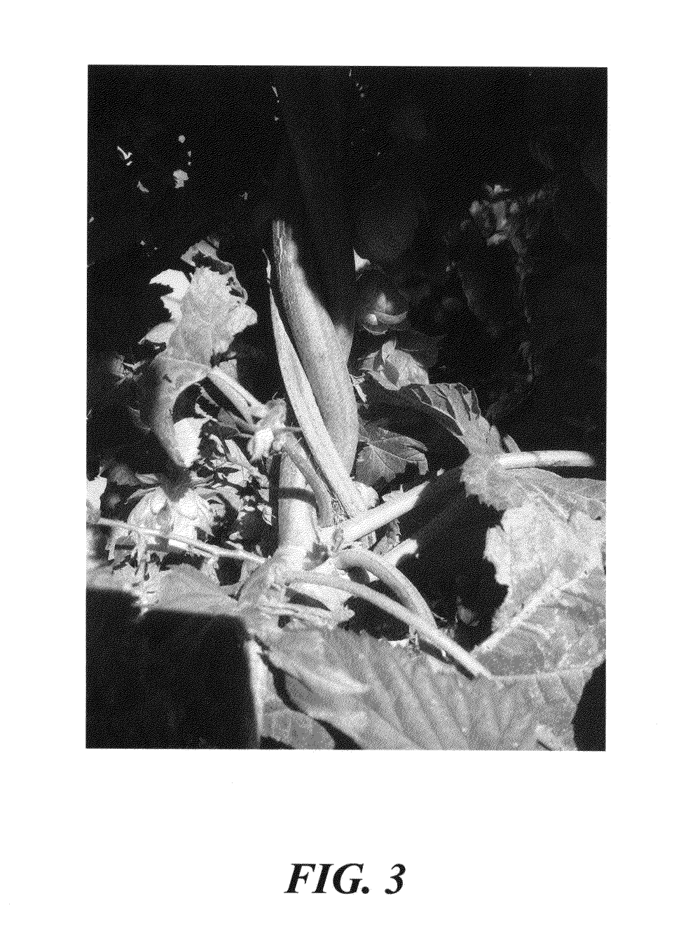

FIG. 4 illustrates the cones and leaves of a mature ADHA 871 hop plant.

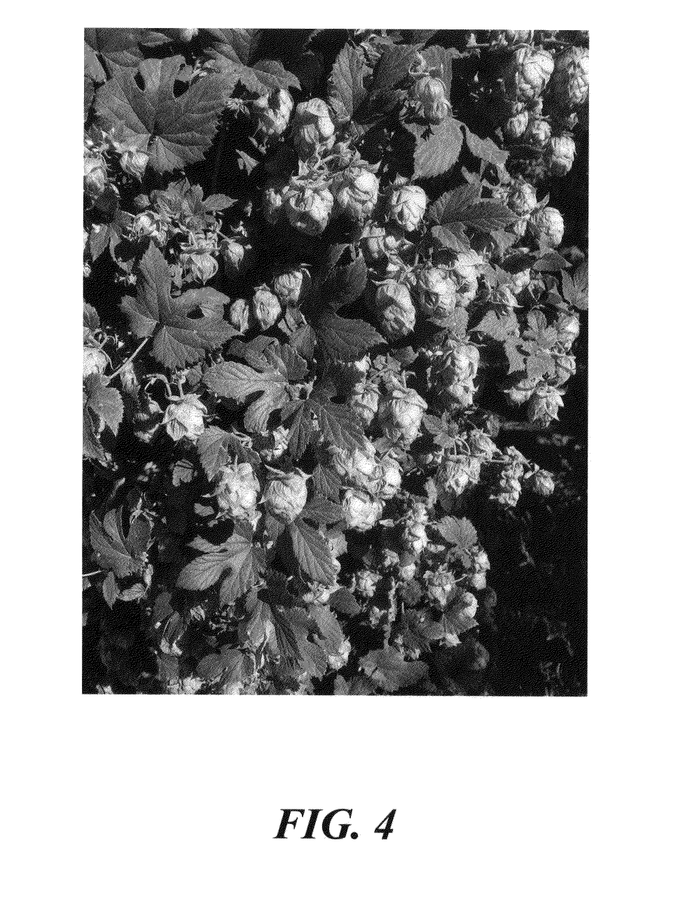

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2013 growing season at Moxee, Wash. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average. The color chart used was The Royal Horticultural Society Colour Chart, Copyright 2001, and all color observations were made outdoors in full sunlight.
Ploidy: Diploid
Use: Brewing beer.
Harvest date: September 15 to 25 during the 2009-2012 growing seasons, Moxee, Wash.
Crop yield: 2200 to 2300 pounds per acre
Disease susceptibility: Tolerant of both downy mildew and powdery mildew.
Pest susceptibility: Average susceptibility to pests, including hop aphids and two spotted spider mites.
Bine:
   Color.—Yellow Green 146A.
   Stripe.—Present, Red Purple 60A.
   Bine diameter.—7.35 mm at base; 9.53 mm at 6 feet; and no measurement taken at terminal end.
Leaf:
   Color of mature leaf upper surface.—Green 147A.
   Color of mature leaf lower surface.—Green 147B.
   Number of lobes.—1-7.
   Margin.—Serrate.
   Serrations per inch.—4-5.
   Petiole color at base.—Yellow Green 144A.
   Vein color.—Green 144A.

1. What is claimed is a new and distinct hop plant as shown and described herein.
